(19) United States
(12) Patent Application Publication
(10) Pub. No.: US 2021/0319898 A1
(43) Pub. Date: Oct. 14, 2021

(54) HEALTH MONITORING SYSTEM HAVING PORTABLE HEALTH MONITORING DEVICES AND METHOD THEREFOR

(71) Applicant: Cardiai Technologies Ltd., Calgary (CA)

(72) Inventors: Anmol Singh KAPOOR, Calgary (CA); Raman KOUL, Calgary (CA); Gang WANG, Calgary (CA); Sidharth BHINDER, Calgary (CA)

(73) Assignee: Cardiai Technologies Ltd., Calgary, AB (CA)

(21) Appl. No.: 17/261,493

(22) PCT Filed: Sep. 5, 2019
(86) PCT No.: PCT/CA2019/051244

§ 371 Date: Jan. 19, 2021

Related U.S. Application Data
(60) Provisional application No. 62/755,084, filed on Nov. 2, 2018.
(60) Provisional application No. 62/727,429, filed on Sep. 5, 2018.

(51) Int. Cl. G16H 50/20; G16H 10/60; G16H 10/20; G16H 10/40; G16H 80/00; G16H 30/20; G16H 40/63; G16H 40/67; A61B 5/0205

(52) U.S. Cl. CPC G16H 50/20; G16H 10/60; G16H 10/20; G16H 10/40; A61B 5/0205; G16H 30/20; G16H 40/63; G16H 40/67; G16H 80/00

(57) ABSTRACT

A health-monitoring system and method use artificial intelligence for monitoring patient health based data acquired from one or more health-monitoring data-sources. The health-monitoring data-sources may include one or more wearable devices, one or more portable health-monitoring devices and other data sources. The health-monitoring system may use artificial intelligence to analyze acquired health data and physiological measurements for monitoring patient health.

### CROSS-REFERENCE TO RELATED APPLICATIONS

This application claims the benefit of U.S. Provisional Patent Application Ser. Nos. 62/727,429, filed Sep. 5, 2018, and 62/755,084, filed Nov. 2, 2018, the content of each of which is incorporated herein by reference in its entirety.

### FIELD OF THE DISCLOSURE

The present disclosure relates generally to a health-monitoring system and method for monitoring patient health, and in particular to a health-monitoring system and method using artificial intelligence for monitoring patient health-based data acquired from one or more health-monitoring data-sources.

### BACKGROUND

Patient-physician “face time” is only a fraction of the actual time that patients spend when they visit their physician's office or a hospital. Patients often spend a significant amount of time completing administrative paperwork, healthcare questionnaires, and in the waiting room before actually meeting face-to-face with the physician, thereby giving rise to a significant burden to patients in monitoring their health conditions and solving their health issues.

On the other hand, doctors in the current health system also face a significant amount of administrative work and need an improved system and method for reducing administrative overhead.

Moreover, it has been observed that a large number of patients' visits with health professionals are for “minor” concerns such as for obtaining test results, health advice, and the like, which consume health system significant resources and reduce the efficiencies thereof.

### SUMMARY

It may be possible for physicians to diagnose certain illnesses without an in-person meeting with a patient if the physician is given access to some basic background health information, which may include a description of symptoms, information from analytes found in the patient's bodily fluids or tissue, blood sugar, electrolytes, and biomarkers of heart function, liver function, and kidney function, endocrine biomarkers, haematological biomarkers, urine biomarkers, and/or the like. Moreover, a significant number of health issues can be resolved if the patients follow basic health advice tailored to their personal needs. If patients are able to communicate this information to physicians over a mobile device or with a computer, then long wait times at and overcrowding of physician's offices and hospitals may be reduced.

There exists a need for point-of-care (POC) devices that are inexpensive, user friendly, sensitive and portable. Moreover, there is a need for a system that reduces the burden on the existing healthcare system and reduces the wait time for patients to access healthcare at clinics and hospitals. A portable POC device may be used in combination with an artificial intelligence (AI) based platform that is available through a computer or by a mobile-device based application to: assess patient health data; filter out frivolous health issues; provide accessible personalized-health management advice to patients; and communicate serious patient-specific health concerns to healthcare providers. Such a system with portable POC devices and an AI platform may be used as a communication and monitoring tool by both physicians and patients, which may streamline access to healthcare and reduce the strain on limited healthcare resources.

Embodiments herein disclose a health-monitoring system and method using artificial intelligence for monitoring patient health-based data acquired from one or more health-monitoring data-sources. The health-monitoring data-sources may include one or more wearable devices, one or more portable health-monitoring devices and other data sources. The health-monitoring system may use artificial intelligence to analyze acquired health data and physiological measurements for monitoring patient health. The wearable device may comprise one or more sensors for measuring a patient's physiological parameters such as heart rate, blood pressure, and/or the like.

The portable health-monitoring device (also denoted as a “point-of-care (POC) device”) may monitor the user's health by monitoring the electrochemical reactions of one or more analytes found in bodily fluids or tissue, such as blood sugar, electrolytes, and biomarkers of heart function, liver function, and kidney function, endocrine biomarkers, haematological biomarkers, urine biomarkers, and/or the like. In some embodiments, the portable health-monitoring device may monitor the user's health using other suitable methods such as fluorescence methods, absorbance spectroscopy, suitable spectroscopic means of analyte recognition, and/or the like.

The wearable devices and portable health-monitoring devices may wirelessly communicate with one or more servers and transmit acquired data thereto.

According to one aspect of this disclosure, there is provided a device and system for tracking and managing patient health data. The system includes using an artificial intelligence (AI) platform to assess health data, provide customized and personal health management advice to patients, and allow health professionals to efficiently and effectively develop and dispense personalized care-plans to patients.

In some embodiments, the AI used in the system solves an important problem of monitoring and assessing physiological and behavioral data from individual patients and providing personalized healthcare plans and advice to patients. The system and/or device may also dispense health-management advice to the patient or instruct the patient to perform one or more self-tests with one or more POC devices. The AI platform assesses the data generated by the POC devices to make a health-management decision or assess the data and evaluate whether the patient requires: home-based health management (e.g., normal health); needs to visit the clinic (e.g., a moderate health risk); needs to get emergency health care; needs to be hospitalized (e.g., a severe health risk). The AI platform then updates the electronic medical record (EMR) of the patient. The system can also provide health professionals with easy access to an individual's health data and facilitate communication between health professionals and patients.

In some embodiments, the system disclosed herein uses point-of-care (POC) device for collecting information from analytes found in samples of a patient's bodily fluids and/or tissue. The POC device includes a portable reader and a cartridge for receiving and analyzing such samples. The cartridge may be an electrochemical sensor strip capable of receiving a sample or alternatively in any other forms suitable for receiving the sample. When a sample is received on the cartridge, the cartridge is then inserted into the reader, which can determine the presence and/or quantity of certain biomarkers.

The users of the POC device are able to avoid making a visit to the clinic or hospital for simple diagnostic tests such as a finger-prick blood test. This reduces the patient wait-time at clinics and time spent by health professionals to perform simple diagnostic tests.

In some embodiments, in order to amplify a biomolecular binding-signal, a cartridge having a nanostructured sensing surface may be used to achieve improved sensitivity.

In some embodiments, the nanostructured sensing surface of the cartridge can be targeted to bind with various biomarkers found in samples of bodily fluids and/or tissue.

According to one aspect of the present disclosure, the system disclosed herein uses an AI platform. The AI platform utilizes a neural network to process and analyze health data input from various sources and produces a personalized assessment of an individual patient's health status.

In some embodiments, the POC device connects wirelessly to the AI platform to transmit data collected from analyzing patient's bodily fluid samples and/or tissue samples that are placed on the cartridge, which comprises an electrochemical sensor strip. Alternatively, the POC device may connect to the AI platform via suitable wired connections.

In some embodiments, the AI platform obtains data wirelessly from various devices, including mobile devices such as phones and wearable devices such as smartwatches, that are capable of monitoring physical activity and physiological health indicators such as but not limited to heart rate, heart rhythm, blood pressure, breathing patterns, blood glucose levels, blood oxygen saturation, hormone levels, other blood biomarkers, and/or the like.

In some embodiments, the AI platform obtains data from the user/patient's EMR, imaging tests run at a clinic or hospital, and the results from one or more laboratory tests.

In some embodiments, the AI platform may prompt the patient to answer a personalized health questionnaire.

In some embodiments, the AI platform may utilize a cloud-based storage system to access and store medical and health data including a patient's EMRs.

In some embodiments, the AI platform may have access to, obtain data from, and be capable of modifying the EMR of the patient.

In some embodiments, the AI platform may analyze physiological data from various devices, personalized health questionnaires, EMRs, imaging tests, laboratory tests and data relating to prescribed medications and make a personalized evaluation of the user/patient's medical condition and/or provide a medical diagnosis.

In some embodiments, the AI platform may utilize a live-chat bot to ask the patient/user personalized questions relating to their health and/or medical status or conditions. In some embodiments, the AI platform may dispense personalized health management and/or medical advice or information to patients/users.

In some embodiments, the AI platform may make a health-management decision for the patient and instruct or suggest them to perform a certain activity such as remaining at their current location, visiting a health care facility, visiting a medical clinic, or visiting a hospital.

In some embodiments, the AI platform may prompt a patient to refill a medical prescription, schedule a medical check-up, and/or schedule or attend other health-related appointments.

In some embodiments, the AI platform may communicate the patient's health or medical condition, status, or other health and/or medical information to a designated health professional.

In some embodiments, the AI platform may communicate a patient's medical and or health condition to emergency-response professionals such as paramedics or law enforcement.

In some embodiments, the AI platform and system disclosed herein is made available to patients and health professionals through a software application capable of running on a mobile computing device such as a laptop computer, tablet or smartphone.

According to one aspect of this disclosure, there is provided a network system for monitoring health conditions of one or more patients. The system comprises one or more health-monitoring data sources for acquiring health-related data of the one or more patients, said one or more data sources comprising at least one personalized health-monitoring device; one or more first client-computing devices associated with and used by the one or more patients; one or more second client-computing devices associated with and used by one or more health professionals; one or more server computers; a secured database; and a network functionally coupled to the one or more health-monitoring data sources, the one or more first client-computing devices, the one or more second client-computing devices, the one or more server computers, and the secured database. The one or more first client-computing devices, the one or more second client-computing devices, and the one or more server computers are configured for collecting health-related data acquired by the one or more health-monitoring data sources; storing the collected health-related data in the secured database as electronic medical records (EMRs); automatically analyzing the collected health-related data and assessing the health conditions of the one or more patients using an artificial intelligence (AI) platform for obtaining health-analysis results and health and medical advices; based on the obtained health-analysis results and assessed health conditions, directing each of the one or more patients for one of managing their health conditions at home, visiting a clinic for health management, and visiting a hospital or emergency room for health management; and updating the EMRs.

In some embodiments, the one or more first client-computing devices, the one or more second client-computing devices, and the one or more server computers are further configured for receiving input from the one or more second client-computing devices; and revising the obtained health-analysis results and health and medical advices based the received input.

In some embodiments, the one or more first client-computing devices, the one or more second client-computing devices, and the one or more server computers are further configured for storing a portion of the EMRs into a personal-data database of each of the one or more first client-computing devices, the portion of the EMRs being related to the patient associated with said first client-computing device.

In some embodiments, each of the one or more first client-computing devices comprises a first live-chat module, and each the one or more second client-computing devices comprising a second live-chat module; and wherein the first and second live-chat modules are communicative with each other via the network.

In some embodiments, the AI platform is configured for using machine learning methods to process and analyze the collected health-related data.

In some embodiments, the AI platform is configured for using a neural network to process and analyze the collected health-related data.

In some embodiments, the collected health-related data comprises a personalized health questionnaire.

In some embodiments, the AI platform is configured for monitoring physical activity and physiological health indicators of at least one of a heart rate, a heart rhythm, a blood pressure, a breathing pattern, a blood glucose level, and a blood oxygen saturation.

In some embodiments, the AI platform is configured for one or more of evaluating the medical conditions of the one or more patients, providing medical diagnoses thereto, providing health management thereto, providing medical advices thereto, and communicating the health or medical information thereof to one or more health professionals and/or emergency-response professionals, based on the analysis of the collected health-related data and assessment of the health conditions of the one or more patients.

In some embodiments, the at least one personalized health-monitoring device each comprises a portable reader and a cartridge for receiving and analyzing samples of bodily fluids and/or tissue if a respective one of the one or more patients.

In some embodiments, the cartridge comprises a nanostructured sensing surface for collecting and amplifying a biomolecular binding-signal of said samples of bodily fluids and/or tissue.

According to one aspect of this disclosure, there is provided a method for monitoring health conditions of one or more patients. The method comprises: collecting health-related data of the one or more patients from one or more health-monitoring data sources, said one or more data sources comprising at least one personalized health-monitoring device; storing the collected health-related data in a secured database as electronic medical records (EMRs); automatically analyzing the collected health-related data and assessing the health conditions of the one or more patients using an artificial intelligence (AI) platform for obtaining health-analysis results and health and medical advices; based on the obtained health-analysis results and assessed health conditions, directing each of the one or more patients for one of managing their health conditions at home, visiting a clinic for health management, and visiting a hospital or emergency room for health management; and updating the EMRs.

In some embodiments, the method further comprises: receiving input from the one or more second client-computing devices; and revising the obtained health-analysis results and health and medical advices based the received input.

In some embodiments, the method further comprises: storing a portion of the EMRs into a personal-data database of each of the one or more first client-computing devices, the portion of the EMRs being related to the patient associated with said first client-computing device.

In some embodiments, the method further comprises: providing a first live-chat module to each of the one or more patients; providing a second live-chat module to each of one or more health professionals; and establishing communication between at least one of the first live-chat modules and at least one of the second live-chat modules via a network.

In some embodiments, the method further comprises: using a neural network in the AI platform to process and analyze the collected health-related data.

In some embodiments, the collected health-related data comprises a personalized health questionnaire.

In some embodiments, the method further comprises: using the AI platform for monitoring physical activity and physiological health indicators of at least one of a heart rate, a heart rhythm, a blood pressure, a breathing pattern, a blood glucose level, and a blood oxygen saturation.

In some embodiments, the AI platform is configured for one or more of evaluating the medical conditions of the one or more patients, providing medical diagnoses thereto, providing health management thereto, providing medical advices thereto, and communicating the health or medical information thereof to one or more health professionals and/or emergency-response professionals, based on the analysis of the collected health-related data and assessment of the health conditions of the one or more patients.

In some embodiments, the at least one personalized health-monitoring device each comprises a portable reader and a cartridge for receiving and analyzing samples of bodily fluids and/or tissue if a respective one of the one or more patients; and said collecting the health-related data comprises: receiving the samples of bodily fluids and/or tissue using the cartridge; analyzing the received samples of bodily fluids and/or tissue using the portable reader; and collecting the health-related data from the portable reader.

In some embodiments, the cartridge comprises a nanostructured sensing surface for collecting and amplifying a biomolecular binding-signal of said samples of bodily fluids and/or tissue.

### BRIEF DESCRIPTION OF THE DRAWINGS

FIG. 1 is a schematic diagram of a personalized health-monitoring system, according to some embodiments of the present disclosure;

FIG. 2 is a schematic diagram showing a simplified hardware structure of a computing device of the personalized health-monitoring system shown in FIG. 1;

FIG. 3 a schematic diagram showing a simplified software architecture of a computing device of the personalized health-monitoring system shown in FIG. 1;

FIG. 4 is a block diagram showing a functional structure of the personalized health-monitoring system shown in FIG. 1;

FIG. 5 shows a non-exhaustive list of functions and characteristics of a patient's app of the personalized health-monitoring system shown in FIG. 1;

FIG. 6 shows a non-exhaustive list of functions and characteristics of a doctor's app of the personalized health-monitoring system shown in FIG. 1;

FIG. 7A shows anon-exhaustive list of physiological measurements that a wearable device of the personalized health-monitoring system shown in FIG. 1 may monitor;

FIG. 7B shows anon-exhaustive list of physiological measurements that a portable health-monitoring device of the personalized health-monitoring system shown in FIG. 1 may monitor;

FIG. 7C shows a non-exhaustive list of a patient's medication records as part of the health-monitoring data-sources of the personalized health-monitoring system shown in FIG. 1;

FIG. 7D shows a non-exhaustive list of a patient's medical imaging records as part of the health-monitoring data-sources of the personalized health-monitoring system shown in FIG. 1;

FIG. 7E shows a non-exhaustive list of a patient's medical test-result records as part of the health-monitoring data-sources of the personalized health-monitoring system shown in FIG. 1;

FIG. 8 shows a secretion cycle of B-type natriuretic peptide (BNP) and N-terminal pro b-type natriuretic peptide (NT-Pro-BNP), obtained from the academic paper entitled “B-Type Natriuretic Peptides and Echocardiographic Measures of Cardiac Structure and Function, JACC” by Richard W. Troughton and A. Mark Richards, and published in Cardiovascular Imaging, Feb. 2009, 2 (2) 216-225;

FIG. 9 shows BNP/NT-ProBNP secretion, obtained from the academic paper entitled “Role of B-type natriuretic peptide (BNP) and NT-proBNP in clinical routine,” by Michael Weber and Christian Hamm, and published in Heart 2006; 92:843-849;

FIGS. 10A to 10C show a nanostructured sensing surface of an electrochemical sensor strip or cartridge coupled to a portable health-monitoring device of the personalized health-monitoring system shown in FIG. 1, the nanostructured sensing surface being targeted to bind to certain analytes found in bodily fluids and/or tissue;

FIG. 11A illustrates a flowchart showing the steps of an AI-assisted process which may be repeatedly executed by a management and decision module of the personalized health-monitoring system shown in FIG. 1, for making medical diagnoses and health management decisions for a patient;

FIG. 11B shows the detail of the AI-assisted management sub-process executed in the AI-assisted process shown in FIG. 11A for making AI-assisted assessment of a patient's health or medical condition, guiding triage, and management of the patient;

FIG. 12 is a schematic representation of a deep neural nets (DNN) based AI method used in the AI-assisted process shown in FIG. 11A;

FIG. 13 is a simplified flowchart showing a process of using the personalized health-monitoring system shown in FIG. 1 for health monitoring;

FIG. 14 shows a list of functions that a patient module of the personalized health management system shown in FIG. 1 may provide to a patient in some embodiments;

FIG. 15 shows a flowchart of the patient's module corresponding to the functions shown in FIG. 14;

FIGS. 16A and 16B show a list of functions that a health-professional module of the personalized health management system shown in FIG. 1 may provide to a doctor in some embodiments;

FIG. 17 shows a flowchart of the health-professional module corresponding to the functions shown in FIGS. 16A and 16B;

FIG. 18 shows a list of functions that a lab module of the personalized health management system shown in FIG. 1 may provide to a lab staff in some embodiments;

FIG. 19 shows a flowchart of the lab module corresponding to the functions shown in FIG. 18;

FIG. 20 shows a list of functions that a pharmacy module of the personalized health management system shown in FIG. 1 may provide to a pharmacy staff in some embodiments;

FIG. 21 shows a flowchart of the pharmacy module corresponding to the functions shown in FIG. 20.

### DETAILED DESCRIPTION

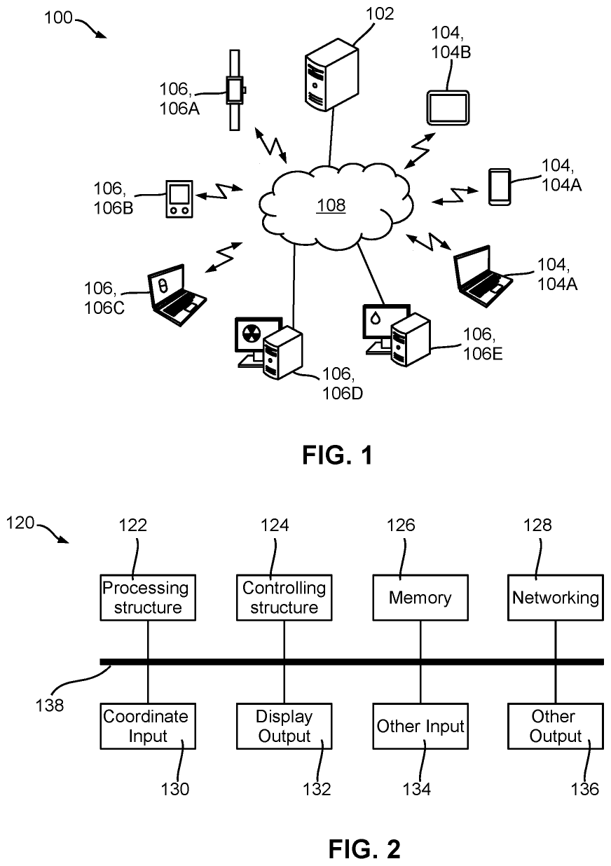

Turning now to FIG. 1, a personalized health-monitoring system is shown and is generally identified using reference numeral 100. The personalized health-monitoring system 100 has at least two types of users, including patients and doctors. Herein, the term “patient” is used for referring to a person or a user using the health-monitoring system 100 for tracking and managing his/her health conditions. Thus, the term “patient” does not necessarily imply that the person has any sickness or health issues. On the other hand, the term “doctor” refers to a user using the health-monitoring system 100 for helping the patient to track and manage the patient's health conditions.

As shown in FIG. 1, the personalized health-monitoring system 100 comprises a server computer 102, a plurality of client computing devices 104, and one or more health-monitoring data-sources 106 functionally interconnected by a network 108, such as the Internet, a local area network (LAN), a wide area network (WAN), a metropolitan area network (MAN), and/or the like, via suitable wired and wireless networking connections.

Depending on the implementation, the one or more health-monitoring data-sources 106 may comprise one or more personalized health-monitoring or health-data-acquisition devices such as wearable health-monitoring devices 106A (e.g., smartwatches) for collecting patients' physiological data (such as heart rates, heart rhythms, blood pressures, breathing patterns, blood glucose levels, and/or the like), analytical health-monitoring devices 106B for analyzing patients' biological samples (e.g., bodily fluid, tissue, and/or the like), portable health-monitoring devices such as portable blood-pressure monitors, and/or similar devices.

The one or more health-monitoring data-sources 106 may alternatively or may also comprise one or more medical records such as medication records 106C collected by patients' and/or doctors' computing devices, medical imaging records 106D collected by medical devices of hospitals and/or medical labs, test-result records 106E such as blood test results conducted by hospitals and/or medical labs, and/or the like. Such computing devices and medical devices for obtaining the medical records 106C to 106E may be part of the system 100 in some embodiments. In some other embodiments, the computing devices and medical devices for obtaining the medical records 106C to 106E may not be part of the system 100. Rather, the system 100 provides a data-source interface for interacting with these computing devices and medical devices and receiving medication records 106C to 106E therefrom.

The server computer 102 executes one or more server programs. Depending on implementation, the server computer 102 may be a server computing device, and/or a general purpose computing device acting as a server computer while also being used by a user.

The client computing devices 104 include one or more client computing devices 104A used by one or more patients and one or more client computing devices 104B used by doctors. Each client computing device 104 executes one or more client application programs (or so-called “apps”) and for users to use. The client computing devices 104 may be portable computing devices such as laptop computers, tablets, smartphones, Personal Digital Assistants (PDAs) and the like. However, those skilled in the art will appreciate that one or more client computing devices 104 may be non-portable computing devices such as desktop computers in some alternative embodiments.

Generally, the computing devices 102 and 104 have a similar hardware structure such as a hardware structure 120 shown in FIG. 2. As shown, the computing device 102/104 comprises a processing structure 122, a controlling structure 124, memory or storage 126, a networking interface 128, coordinate input 130, display output 132, and other input and output modules 134 and 136, all functionally interconnected by a system bus 138.

The processing structure 122 may be one or more single-core or multiple-core computing processors such as INTEL® microprocessors (INTEL is a registered trademark of Intel Corp., Santa Clara, Calif., USA), AMD® microprocessors (AMD is a registered trademark of Advanced Micro Devices Inc., Sunnyvale, Calif., USA), ARM® microprocessors (ARM is a registered trademark of Arm Ltd., Cambridge, UK) manufactured by a variety of manufactures such as Qualcomm of San Diego, Calif., USA, under the ARM® architecture, or the like.

The controlling structure 124 comprises one or more controlling circuits, such as graphic controllers, input/output chipsets and the like, for coordinating operations of various hardware components and modules of the computing device 102/104.

The memory 126 comprises a plurality of memory units accessible by the processing structure 122 and the controlling structure 124 for reading and/or storing data, including input data and data generated by the processing structure 122 and the controlling structure 124. The memory 126 may be volatile and/or non-volatile, non-removable or removable memory such as RAM, ROM, EEPROM, solid-state memory, hard disks, CD, DVD, flash memory, or the like. In use, the memory 126 is generally divided to a plurality of portions for different use purposes. For example, a portion of the memory 126 (denoted as storage memory herein) may be used for long-term data storing, for example, storing files or databases. Another portion of the memory 126 may be used as the system memory for storing data during processing (denoted as working memory herein).

The networking interface 128 comprises one or more networking modules for connecting to other computing devices or networks through the network 108 by using suitable wired or wireless communication technologies such as Ethernet, WI-FI®, (WI-FI is a registered trademark of Wi-Fi Alliance, Austin, Tex., USA), BLUETOOTH® (BLUETOOTH is a registered trademark of Bluetooth Sig Inc., Kirkland, Wash., USA), ZIGBEE® (ZIGBEE is a registered trademark of ZigBee Alliance Corp., San Ramon, Calif., USA), 3G, 4G and/or 5G wireless mobile telecommunications technologies, and/or the like. In some embodiments, parallel ports, serial ports, USB connections, optical connections, or the like may also be used for connecting other computing devices or networks although they are usually considered as input/output interfaces for connecting input/output devices.

The display output 132 comprises one or more display modules for displaying images, such as monitors, LCD displays, LED displays, projectors, and the like. The display output 132 may be a physically integrated part of the computing device 102/104 (for example, the display of a laptop computer or tablet), or may be a display device physically separate from, but functionally coupled to, other components of the computing device 102/104 (for example, the monitor of a desktop computer).

The coordinate input 130 comprises one or more input modules for one or more users to input coordinate data, such as touch-sensitive screen, touch-sensitive whiteboard, trackball, computer mouse, touch-pad, or other human interface devices (HID) and the like. The coordinate input 130 may be a physically integrated part of the computing device 102/104 (for example, the touch-pad of a laptop computer or the touch-sensitive screen of a tablet), or may be a display device physically separate from, but functionally coupled to, other components of the computing device 102/104 (for example, a computer mouse). The coordinate input 130, in some implementation, may be integrated with the display output 132 to form a touch-sensitive screen or touch-sensitive whiteboard.

The computing device 102/104 may also comprise other input 134 such as keyboards, microphones, scanners, cameras, Global Positioning System (GPS) component, and/or the like.

The computing device 102/104 may further comprise other output 136 such as speakers, printers and/or the like.

The system bus 138 interconnects various components 122 to 136 enabling them to transmit and receive data and control signals to/from each other.

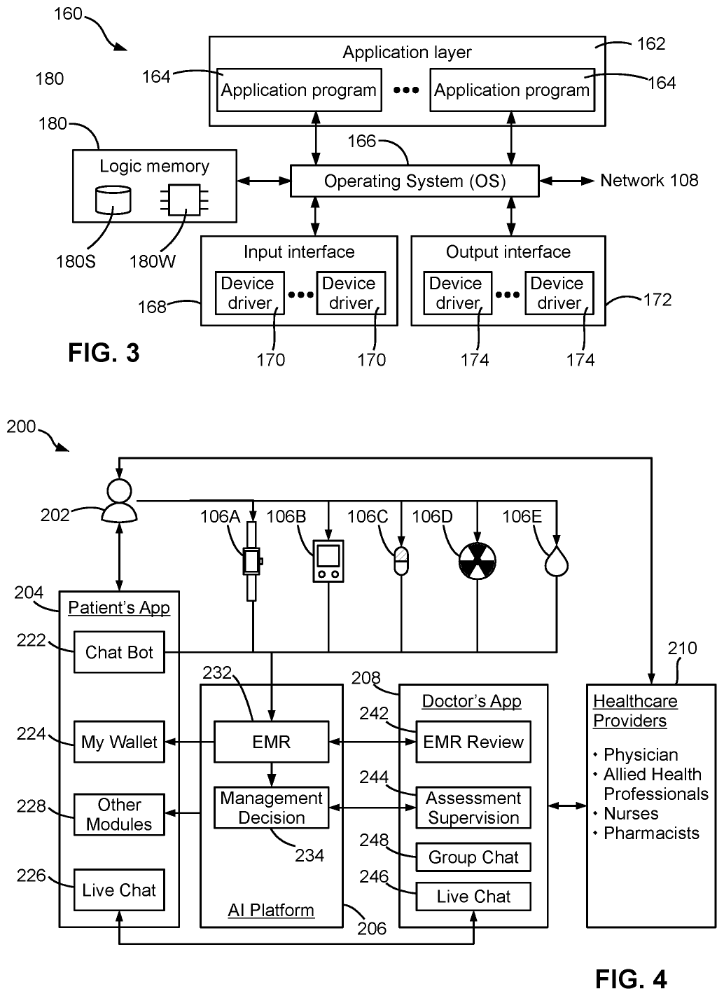

FIG. 3 shows a simplified software architecture 160 of the computing device 102 or 104. The software architecture 160 comprises an application layer 162, an operating system 166, an input interface 168, an output interface 172, and logic memory 180. The application layer 162 comprises one or more application programs 164 executed by or run by the processing structure 122 for performing various tasks. The operating system 166 manages various hardware components of the computing device 102 or 104 via the input interface 168 and the output interface 172, manages logic memory 180, and manages and supports the application programs 164. The operating system 166 is also in communication with other computing devices (not shown) via the network 108 to allow application programs 164 to communicate with those running on other computing devices. As those skilled in the art will appreciate, the operating system 166 may be any suitable operating system such as MICROSOFT® WINDOWS® (MCROSOFT and WINDOWS are registered trademarks of the Microsoft Corp., Redmond, Wash., USA), APPLE® OS X, APPLE® iOS (APPLE is a registered trademark of Apple Inc., Cupertino, Calif., USA), Linux, ANDROID® (ANDRIOD is a registered trademark of Google Inc., Mountain View, Calif., USA), or the like. The computing devices 102 and 104 of the personalized health-monitoring system 100 may all have the same operating system, or may have different operating systems.

The input interface 168 comprises one or more input device drivers 170 for communicating with respective input devices including the coordinate input 130. The output interface 172 comprises one or more output device drivers 174 managed by the operating system 166 for communicating with respective output devices including the display output 132. Input data received from the input devices via the input interface 168 is sent to the application layer 162, and is processed by one or more application programs 164. The output generated by the application programs 164 is sent to respective output devices via the output interface 172.

The logical memory 180 is a logical mapping of the physical memory 126 for facilitating the application programs 164 to access. In this embodiment, the logical memory 180 comprises a storage memory area (180S) that is usually mapped to non-volatile physical memory such as hard disks, solid state disks, flash drives, and the like, generally for long-term data storage therein. The logical memory 180 also comprises a working memory area (180W) that is generally mapped to high-speed, and in some implementations volatile, physical memory such as RAM, generally for application programs 164 to temporarily store data during program execution. For example, an application program 164 may load data from the storage memory area 180S into the working memory area 180W, and may store data generated during its execution into the working memory area 180W. The application program 164 may also store some data into the storage memory area 180S as required or in response to a user's command.

In a server computer 102, the application layer 162 generally comprises one or more server-side application programs 164 which provide server functions for managing network communication with client computing devices 104 and facilitating collaboration between the server computer 102 and the client computing devices 104. Herein, the term “server” may refer to a server computer 102 from a hardware point of view or a logical server from a software point of view, depending on the context.

FIG. 4 is a block diagram showing a functional structure 200 of the personalized health-monitoring system 100. As shown, the functional structure 200 of the personalized health-monitoring system 100 comprises a patient's app 204 which is a client-side application program 164 running on a client computing device 104A of the patient 202, a cloud-based health-monitoring platform 206 using artificial intelligence (AI) and comprising a set of server-side application programs 164 running on a server computer 102, and a doctor's app 208 which is a client-side application program 164 running on a client computing device 104B of the doctor (not shown).

In these embodiments, the personalized health-monitoring system 100 is a subscription-based system to patients 202. Accordingly, the patient's app 204 comprises necessary function modules (not shown) for subscribing the service of the system 100, logging-in, authentication, and the like. The patient's app 204 also comprises a user-interaction module (denoted as a “ChatBot”) 222, a secured personal-data storage (denoted as “My Wallet”) 224, a live-chat module 226, and other modules 228.

The user-interaction module 222 provides the patient 202 a customized questionnaire comprising a plurality of health survey-questions for surveying the general health conditions and life style of the patient 202. The patient may go through the questionnaire and provide their answer to the health survey questions thereof. The system 100 repeatedly update the questionnaire to adapt the questionnaire to the patient's particular health conditions.

The secured personal-data storage 224 in these embodiments is a database with enhanced security for safely storing the patient's personal and health data.

As will be described later, the live-chat module 226 establishes and maintains real-time multimedia communication (e.g., in text, image, voice, and video) with the doctor's app 208 of the patient's doctor. The live-chat modules 226 and 246 may also comprise a suitable file-transfer function for transferring files therebetween. In some embodiments, the live-chat module 226 also provides a group-chat function allowing a plurality of patients 202 to use their patient's apps 204 to chat with one or more doctors via the doctor's apps 208. In some embodiments, the group-chat function may be implemented as a separate function module.

The patient's app 204 also comprises other modules 228 as needed. For example, in some embodiments, the other modules 228 may include a medication-list module for the patient to submit their prescribed and/or current medication list to the doctor, a self-management module for the patient to manage their health-related items such as activities, diets, and the like and also to manage the settings of the patient's app 204, and a news feed module for sending, sharing, receiving, and saving news and posts.

The health-monitoring platform 206 comprises a health database 232 for storing the personal and health data of the patient 202 as electronic medical records (EMRs) and a management and decision module 234. The management and decision module 234 automatically analyzes the health data (collected by the health-monitoring data-sources 106 and stored in the health database 232 as EMRs) and assess the health conditions of the one or more patients 202 for obtaining health-analysis results and corresponding health and medical advices or suggestions.

The doctor's app 208 comprises an EMR review module 242 for reviewing the health data of the patient 202 stored in the health database 232, an assessment supervision module 244 for reviewing, modifying, confirming, and/or forming personalized health and/or medical advices, a live-chat module 246 for communicating with the live-chat module 226 of the patient's app 204, and other modules 248 such as a module for staff management. In these embodiments the doctor's app 208 also comprises an interface (not shown) for communicating and interacting with healthcare providers 210 such as other physicians, allied health professionals, pharmacists, and/or the like, to facilitate the healthcare providers 210 in providing services to the patient 202.

In these embodiments, the EMR review module 242 may receive the doctor's input and instruct the server 102 to revise the health-analysis results and corresponding health and medical advices or suggestions obtained by the management and decision module 234.

In some embodiments, the live-chat module 246 may also provide a group-chat function allowing the doctor to chat with a plurality of patients. In some embodiments, the live-chat module 246 may further allow group chat with other users such as between doctors, between doctors and staff, between staff members, between doctors and other health providers, and/or the like. In some embodiments, the group-chat function with other users may be controlled by the user/doctor of the doctor's app 208 and/or the system policy settings such that the ability of initiating a group chat from certain users is restricted. For example, in some embodiments, a group chat between doctors and staff members may be initiated by a doctor, but is not allowed to be initiated by a staff member.

With the functional structure 200 shown in FIG. 4, the user-interaction module collects answers of the questionnaire from the patient 202 and sends collected questionnaire answers to the health-monitoring platform 206 for establishing or updating the patient's personal and health data. Specifically, the health-monitoring platform 206 associates the received health data with the patient 202 and stores received health data in the health database 232 as EMRs.

The one or more health-monitoring data-sources 106 such as the wearable health-monitoring devices 106A, analytical health-monitoring devices 106B, medication records 106C, medical imaging records 106D, test-result records 106E, and the like, also transmit health data of the patient 202 to the health-monitoring platform 206. The health-monitoring platform 206 associates the received health data with the patient 202 and stores received health data in the health database 232 as EMRs.

After the EMRs of the patient 202 are established or updated, the EMRs are also sent to the patient's app 204 and stored in the secured storage thereof. A notification may also be sent to the patient's app 204 to notify the patient that his/her health data has been updated.

When the patient's health data is updated or upon a request from the patient's doctor, the health-monitoring platform 206 also sends the EMRs of the patient 202 to the EMR review module 242 of the doctor's app used by the patient's doctor.

The management decision module 234 of the health-monitoring platform 206 automatically accesses the health database 232 and uses a suitable AI method to analyze the health data (i.e., EMRs) of the patient 202 to determine the patient's health conditions and corresponding health and medical advices or suggestions. The analytical results and advices (collectively denoted as “health results” hereinafter) determined by the management decision module 234 are sent to the doctor's app 208 such that the doctor may use the assessment supervision module 244 of the doctor's app 208 to review, modify, confirm, and/or form personalized health and/or medical advices. When needed, the doctor may use the assessment supervision module 244 of the doctor's app 208 to query the management decision module 234 for reviewing, modifying, confirming, and/or forming personalized health and/or medical advices.

In these embodiments, the health results (in which the advices may be modified by the patient's doctor as described above) are not directly sent to the patent's app 204. Rather, the doctor and the patient 202 may use their live-chat modules 246 and 226 of the doctor's app 208 and the patient's app 204, respective to communicate with respect to the health results. The doctor may use the file-transfer function of the live-chat module 246 to send the health results or a portion thereof to the patient 202. Therefore, the patient 202 does not need to visit the doctor's office for obtaining the health results.

In these embodiments, a live chat may only be initiated by the live-chat module 246 of the doctor's app 208. Of course, those skilled in the art will appreciate that in some alternative embodiments, the live-chat module 226 of the patient's app 208 may also initiate a live chat with the live-chat module 246 of the doctor's app 208.

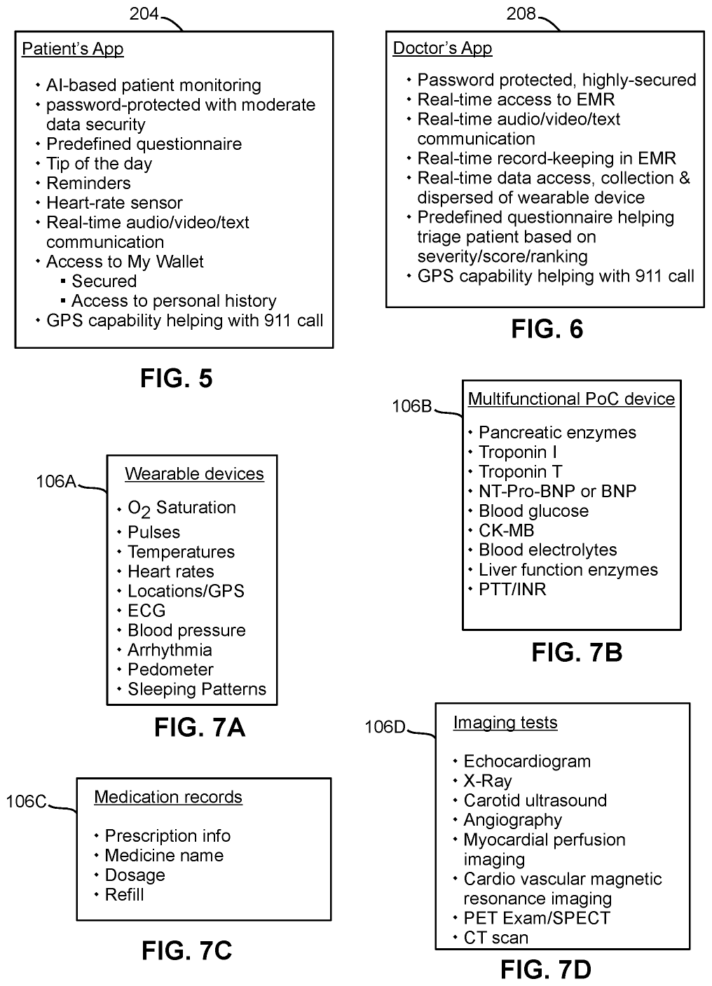

In various embodiments, the patient's app 204 and the doctor's app 208 may provide a plurality of functions. For example, FIG. 5 shows a non-exhaustive list of functions and characteristics of patient's app 204, including:
AI based patient monitoring;
being a password-protected app with moderate data security for ease of use and for lowered cost;
comprising predefined and repeatedly-updated questionnaire for the patient 202 to answer;
showing “Tip of the day” to the patient 202;
providing reminders to the patient 202;
coupling to health-monitoring devices 106 for obtaining real-time health data such as heart-rate;
real-time audio/video/text communication with doctor's app 208, wherein in some embodiments, the patient's app is allowed to send and receive text and/or image messages and to receive audio/video messages, and is not allowed to send audio/video messages;
secured access to the secured personal-data storage 224 (i.e., My Wallet) with access to personal health history; and
leveraging the GPS function of the patient's computing device 104 (such as a smartphone) for helping with 911 calls.

FIG. 6 shows a non-exhaustive list of functions and characteristics of doctor's app 208, including:
password-protected and highly-secured for data security and privacy protection; real-time access to EMRs in the health database 232;
real-time audio/video/text communication (both sending and receiving);
real-time record-keeping in the health database 232;
real-time data access, collection & dispersal of patient's wearable devices;
predefined questionnaire helping triage patient based on severity/score/ranking; and
GPS capability helping with 911 calls.

As described above, the medical data sources 106 may acquire a variety of health data, medical records, and physiological measurements of the patient 202 from a plurality of sources for the system 100 to analyze, monitor, and determine health advices accordingly.

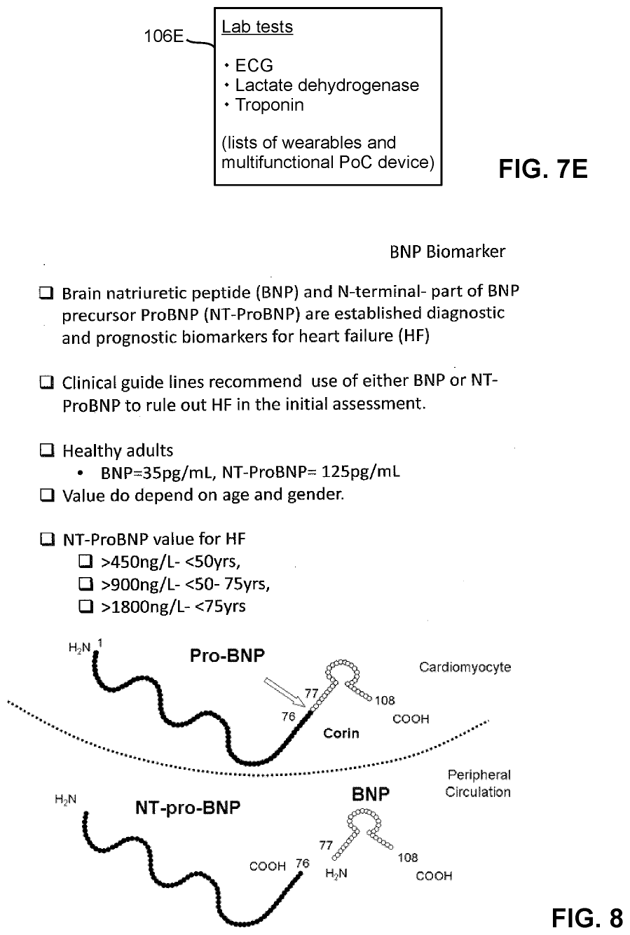

For example, as shown in FIG. 7A, the wearable health-monitoring devices 106A may be used for determining a plurality of physiological measurements including but not limited to:
oxygen saturation;
pulses;
temperatures;
heart rates;
locations (such as using GPS);
electrocardiogram (ECG);
blood pressure;
arrhythmia;
steps/pedometer; and
sleeping patterns.

As shown in FIG. 7B, the analytical health-monitoring devices 106B may be used for determining one or more biomarkers (described in more detail later) including but not limited to:
Pancreatic enzymes;
Troponin I;
Troponin T;
NT-pro-BNP or BNP;
Blood Glucose;
Creatine kinase—muscle/brain (CK-MB);
Blood Electrolytes;
Liver Function Enzymes; and
Partial Thomboplastin time/International normalized time (PT/INR).

As shown in FIG. 7C, the patient's medication records 106C may include:
prescription information (doctor's name, prescription date, and the like);
medicine name;
dosage;
usage log (date/time of taking the medicine, missing a dose, and the like); and
refill information (refillable or not, total number of prescribed refills, total number of used refills, and the like).

Some medication records 106C such as the usage log may be input by the patient 202 via the patient's computing device 104. Some other medication records such as prescription information, medicine name, dosage, and refill information may be input by the doctor and/or pharmacist via their computing devices 104.

As shown in FIG. 7D, the medical imaging records 106D may include (without limited to):
Echocardiogram;
X-Ray (such as Chest X-ray, Posterior anterior, lateral);
Carotid ultrasound;
Angiography;
Myocardial perfusion imaging;
Cardio vascular magnetic resonance imaging;
positron emission tomography (PET) Exam, single-photon emission computerized tomography (SPECT); and
computed tomography (CT) scan.

As shown in FIG. 7E, the medical test-result records 106E may include (without limited to):
ECG;
Lactate dehydrogenase;
Troponin; and
items shown in the list of FIGS. 7A and 7B.

In some embodiments, an analytical health-monitoring device 106B may be coupled to an electrochemical sensor structure (such as in the form of a strip or cartridge) having a bio-sample of the patient 202 for determining the patient's health conditions by detecting various biomarkers in the bio-sample. For example, FIG. 8 shows a secretion cycle of B-type natriuretic peptide (BNP) and N-terminal pro b-type natriuretic peptide (NT-Pro-BNP), obtained from the academic paper entitled “B-Type Natriuretic Peptides and Echocardiographic Measures of Cardiac Structure and Function, JACC” by Richard W. Troughton and A. Mark Richards, and published in Cardiovascular Imaging, Feb 2009, 2 (2) 216-225; DOI:10.1016/j.jcmg.2008.12.006, the content of which is incorporated herein by reference in its entirety. The NT-pro-BNP may be used as a biomarker for indicating heart failure.

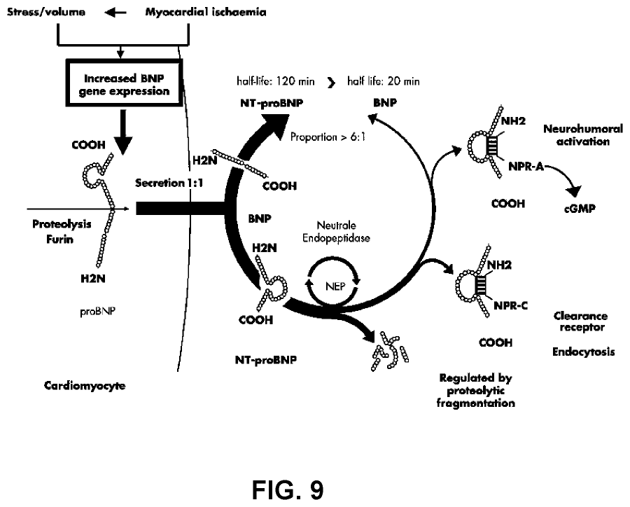

FIG. 9 shows BNP/NT-ProBNP secretion, obtained from the academic paper entitled “Role of B-type natriuretic peptide (BNP) and NT-proBNP in clinical routine,” by Michael Weber and Christian Hamm, and published in Heart 2006; 92:843-849; DOI:10.1136/hrt.2005.071233 the content of which is incorporated herein by reference in its entirety.

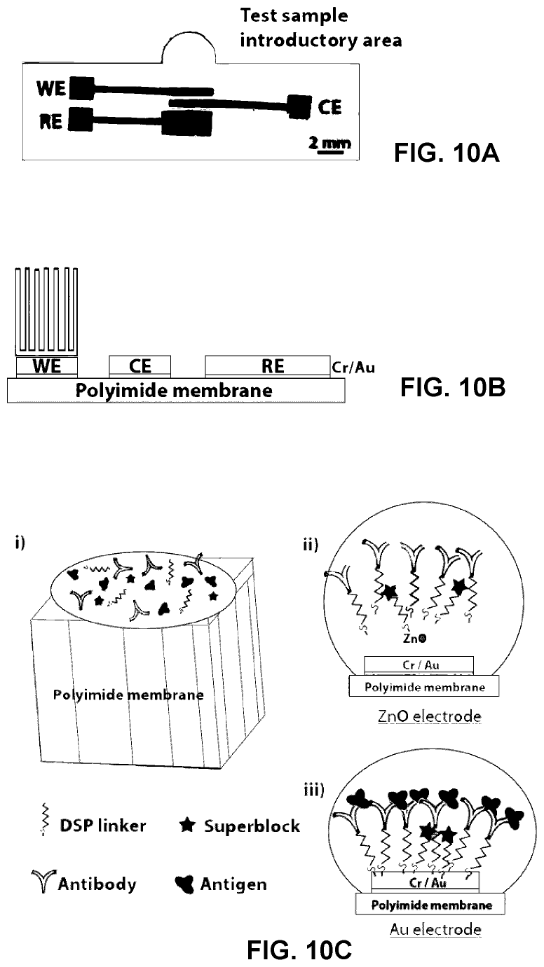

FIGS. 10A to 10C show a nanostructured sensing surface of an electrochemical sensor strip or cartridge targeted to bind to certain analytes found in bodily fluids and/or tissue. FIGS. 10A to 10C are obtained from the academic paper entitled “Ultrasensitive and low-volume point-of-care diagnostics on flexible strips—a study with cardiac troponin biomarkers,” by Nandhinee R. Shanmugam, Sriram Muthukumar, and Shalini Prasad, and published in Sci Rep. 2016; 6: 33423; DOI: 10.1038/srep33423, the content of which is incorporated herein by reference in its entirety.

In some embodiments, the analytical health-monitoring devices 106B may use a testing structure similar to that shown in in FIGS. 10A to 10C for detecting biomarkers. For example, the nanostructured sensing surface of the cartridge may be targeted to bind to NT-pro-BNP for detecting heart failure.

With above-described data obtained from the health-monitoring data-sources 106, the management and decision module 234 may analyze the health conditions of the patient 202 and provide health-analysis results and corresponding health and medical advices or suggestions.

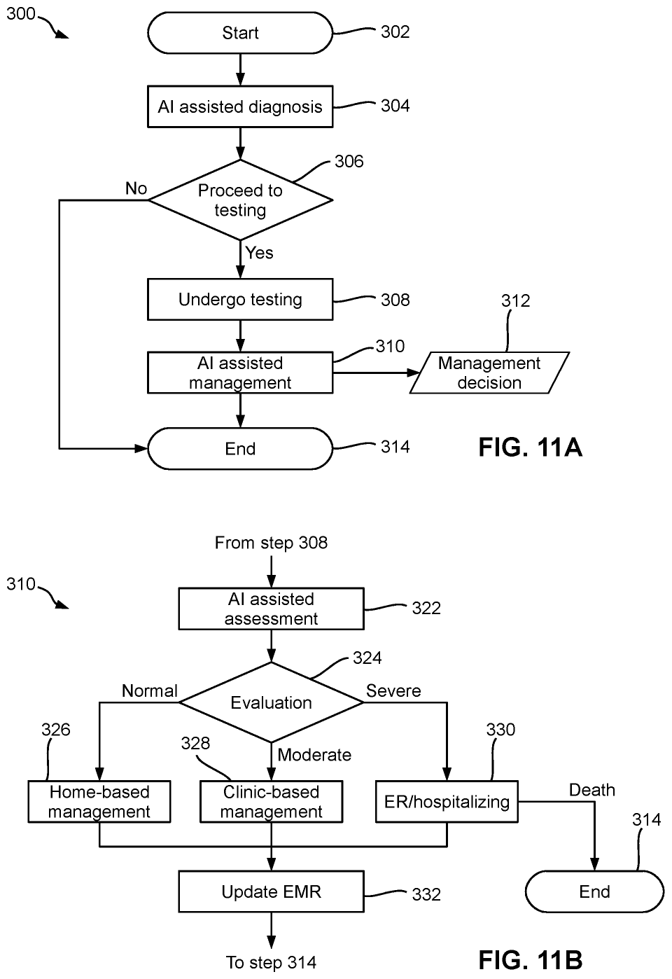

FIG. 11A illustrates a flowchart showing the steps of an AI-assisted process 300 which may be repeatedly executed by the management and decision module 234 for making medical diagnoses and health management decisions for the patient 202.

After the process 300 starts (step 302), an AI-assisted diagnosis is conducted (step 304). Based on the diagnosis, the management and decision module 234 checks if any testing is required (step 306). If testing is not required, the process 200 is terminated (step 314). Otherwise, if testing is required, the testing is then conducted (step 308). The testing results are processed/analyzed by an AI-assisted management sub-process (step 310) for making a management decision (block 312). The process is then terminated (step 314).

FIG. 11B shows the detail of the AI-assisted management sub-process executed at step 310 for making AI-assisted assessment of a patient's health or medical condition, guiding triage and management of the patient.

As shown, an AI-assisted assessment of the patient's health data is first conducted (step 322). Then, an evaluation of the patient's health conditions is conducted based on the assessment result (step 324). If the evaluation result indicates that the patient 202 only requires home-based health management (e.g., in normal health conditions), the patient 202 is directed for a home-based health management for managing his/her health conditions at home (step 326) and the patient's EMRs may be updated (step 332).

If at step 324, the evaluation result indicates that the patient 202 requires to visit his/her doctor at the clinic (e.g., at moderate health risk), the patient 202 is directed for a clinic-based health management for visiting a clinic (such as seeing the doctor) for managing his/her health conditions (step 328) and the EMR may be updated (step 332).

If at step 324, the evaluation result indicates that the patient requires to visit an emergency room (ER) or hospital (e.g., at sever health risk), a corresponding ER or hospital is contacted to send the patient thereof (step 330), and the patient's EMR may be updated (step 332). The process 300 may be repeated until it is no longer required such as at the death of the patient (step 314).

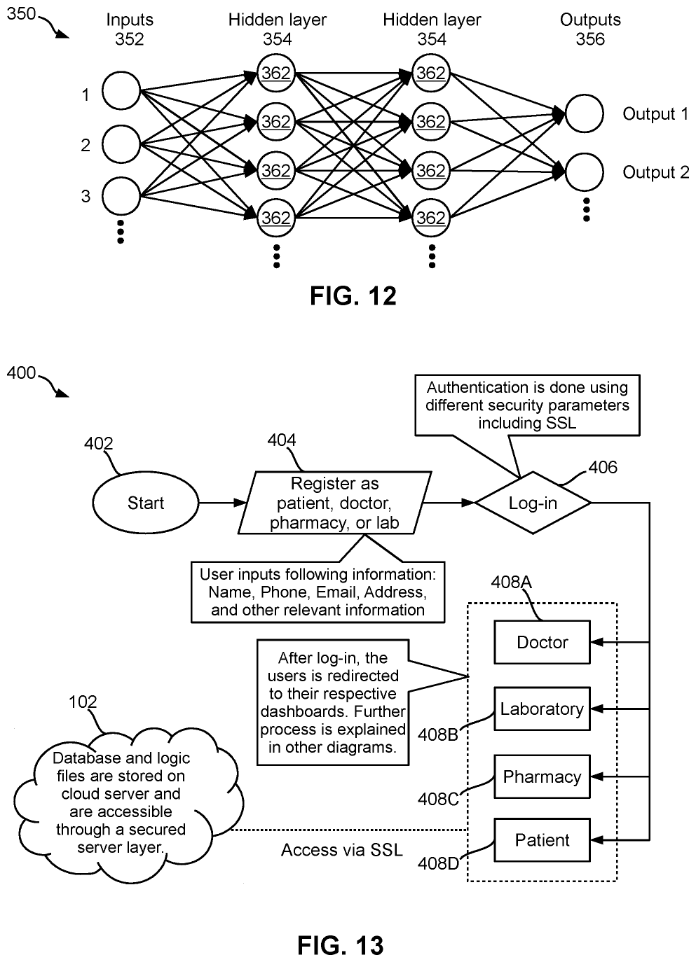

In these embodiments, the personalized health-monitoring system 100 uses deep neural nets (DNN) for deep learning and for analyzing the patient's health data. FIG. 12 is a schematic representation of a DNN-based AI method 350 which comprises a plurality of cascaded, hidden layers 354 having interconnected nodes 362. The hidden layers receive and processes data inputs 352 for generating the outputs 356.

With above-described modules and functions, the personalized health-monitoring system 100 allows users to access the system, obtain health-related data and monitor patients' health conditions. In some embodiments, the users may be patients and doctors. In some other embodiments, the users may also include other health professionals such as medical-lab staff and pharmacists.

FIG. 13 is a simplified flowchart showing a process 400 of using the personalized health-monitoring system 100 for health monitoring.

After the process 400 starts (step 402), a first-time user of the system 100 may register himself/herself with a proper role such as a patient, a doctor, a pharmacist or pharmacy staff, or a lab staff (step 404). At this step the first-time user may need to provide necessary information such as name, phone number, email address, mailing address, and the like.

After registration, the user may log-in to the system 100 (step 406). User-authentication is conducted at this step using necessary security parameters such as Secure Sockets Layer (SSL) for communication with the server 102 (or so-called “cloud”) in which databases and logic files are stored. In these embodiments, the server 102 and the databases and logic files stored therein are accessible via SSL.

After log-in, the user is redirected to a respective dashboard user-interface (UI) 408A to 408D (described in more detail later) based on the user's role and the user may conduct various health-monitoring tasks as described below.

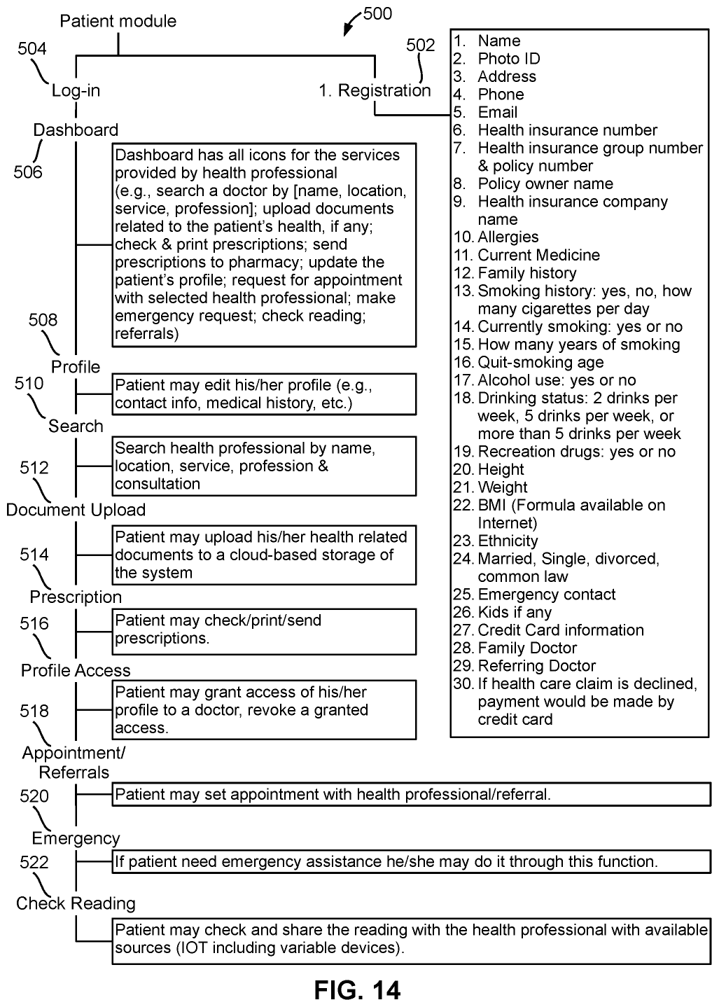

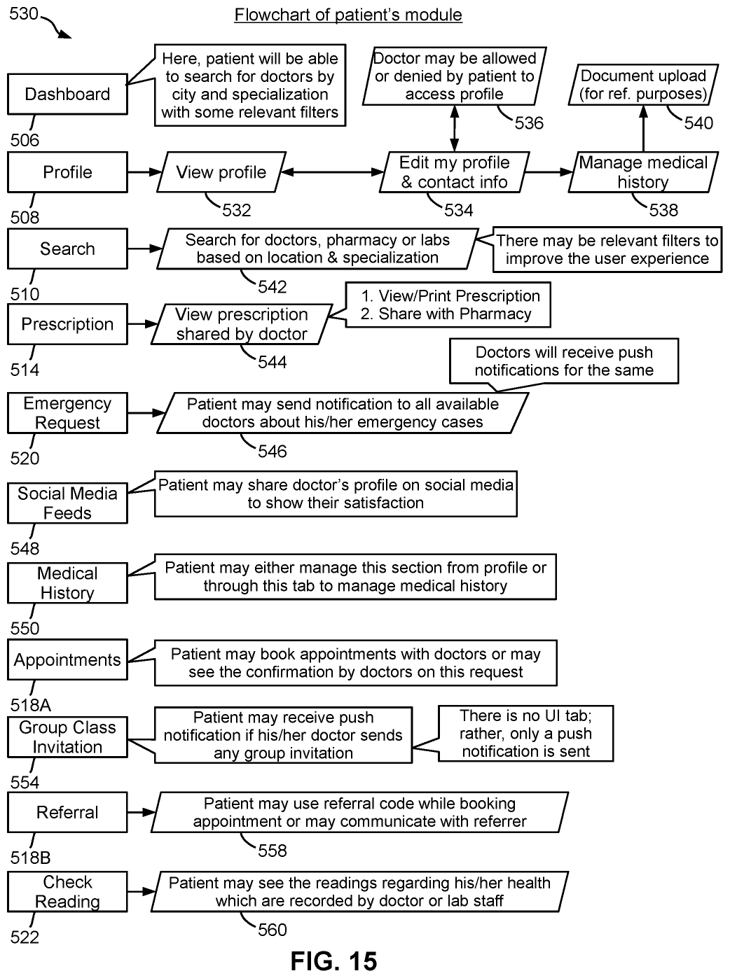

FIG. 14 shows a list of functions that a patient module 500 of the personalized health management system 100 (i.e., a combination of the patient's app 204 and corresponding program modules on the server 102) may provide to the patient 202 in some embodiments. FIG. 15 shows a flowchart 530 of the patient's module 500.

As shown, the patient module provides a registration function 502 for collecting various information of the patient 202 such as name, photo ID, address, phone number, email address, health-insurance number, health-insurance group number and policy number, policy owner's name, health-insurance company name, allergies, current medicine, family history, smoking history (yes, no, or how many cigarettes per day), currently smoking status (yes or no), how many years of smoking, quit-smoking age, alcohol use (yes or no), drinking status (e.g., 2 drinks per week, 5 drinks per week, or more than 5 drinks per week), use of recreation drugs (yes or no), height, weight, Body Mass Index (BMI; with BMI formula available on Internet), ethnicity, marriage status (married, single, divorced, common law), emergency contact, kids (if any), credit card information, family doctor information, referring doctor, payment information (e.g., if health care claim declined, payment would be made by credit card), and/or the like.

After registration 502, the patient 202 is then allowed to log-in (504) to the system 100. A dashboard 506 is then displayed via the patient's app 204 which comprises various icons for the services provided by the system 100 and the health professional (e.g., the doctor).

The patient 202 may use the dashboard 506 to conduct various tasks. For example, the patient 202 may view his/her profile 508 (such as contact information) stored in the system 100 (step 532 of FIG. 15) and edit the profile (step 534) wherein the patient 202 may adjust the access rights 516 of his/her profile such as granting the access to a doctor, revoking a granted access, and/or the like (step 536). The patient 202 may also use the dashboard 506 to manage his/her medical history (step 538). The patient 202 may use the dashboard 506 to upload his/her health-related documents 512 to the system 100 such as to the health database 232 thereof (step 540). For ease of use, the patient's app 204 may comprise a “Profile” tab or menu item on the user interface (UI) displayed thereon.

The patient 202 may use the dashboard 506 to conduct a search 510 for finding a health professional (such as a doctor, a pharmacist, a lab staff) by using a combination of name, location, service, profession, specialization, consultation, and/or the like (step 542). The patient 202 may set up relevant filters for the search. For ease of use, the patient's app 204 may comprise a “Search” tab or menu item on the UI displayed thereon.

The patient 202 may use the dashboard 506 to view prescriptions 514 shared by the doctor (step 544), print prescriptions, and/or send prescriptions to pharmacy. For ease of use, the patient's app 204 may comprise a “Prescription” tab or menu item on the UI displayed thereon.

The patient 202 may use the dashboard 506 to book an appointment 518A with a health professional using a referral 518B (if available). In particular, the patient 202 may use a referral code in booking an appointment or may communicate with referrer (step 558). For ease of use, the patient's app 204 may comprise an “Appointment” tab or menu item on the UI displayed thereon.

The patient 202 may use the dashboard 506 to make an emergency request 520 if the patient needs any emergency assistance. In particular, the patient 202 may send notification to all available doctors about his/her emergency cases (step 546). For ease of use, the patient's app 204 may comprise an “Emergency” tab or menu item on the UI displayed thereon.

The patient 202 may use the dashboard 506 to check his/her health data 522 such as readings of the health-monitoring device 106 and share the health data with health professionals. In particular, the patient 202 may read his/her health readings recorded by a wearable device, a portable health-monitoring device, a doctor, or lab staff (step 560). For ease of use, the patient's app 204 may comprise a “Readings” tab or menu item on the UI displayed thereon.

The patient 202 may use the dashboard 506 to publish social media feeds. For example, the patient 202 may share a doctor's profile on social media to show their satisfaction (step 548). For ease of use, the patient's app 204 may comprise a “Social Media” tab or menu item on the UI displayed thereon.

The patient 202 may use the dashboard 506 to manage his/her medical history (step 550). For ease of use, the patient's app 204 may comprise a “Medical history” tab or menu item on the UI displayed thereon. Alternatively, the patient 202 may manage his/her medical history in his/her profile.

The patient 202 may use the dashboard 506 to receive a group class invitation (described later) from a doctor (step 554). In these embodiment, the patient's app 204 does not provide a tab or menu item on the UI. Rather, a push notification may be sent to the patient's app 204 for notifying the patient 202.

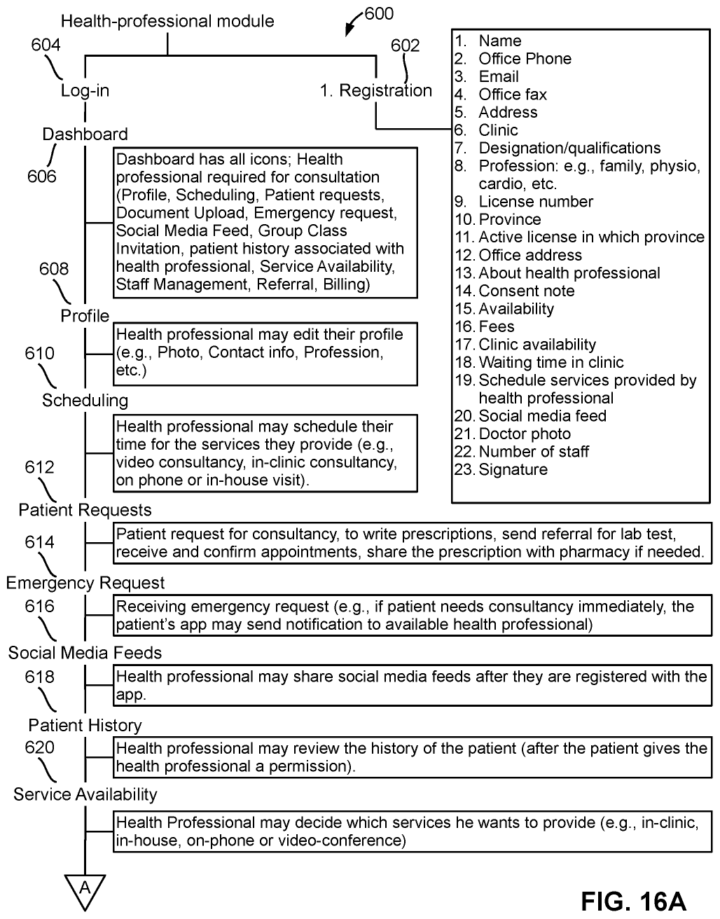

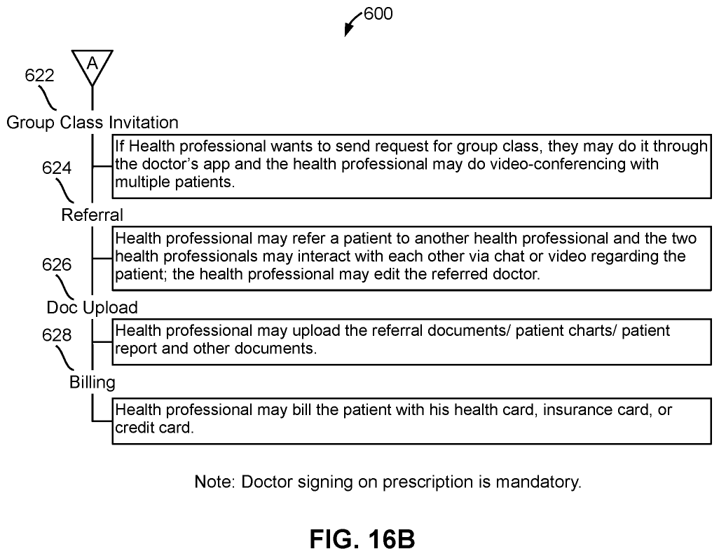

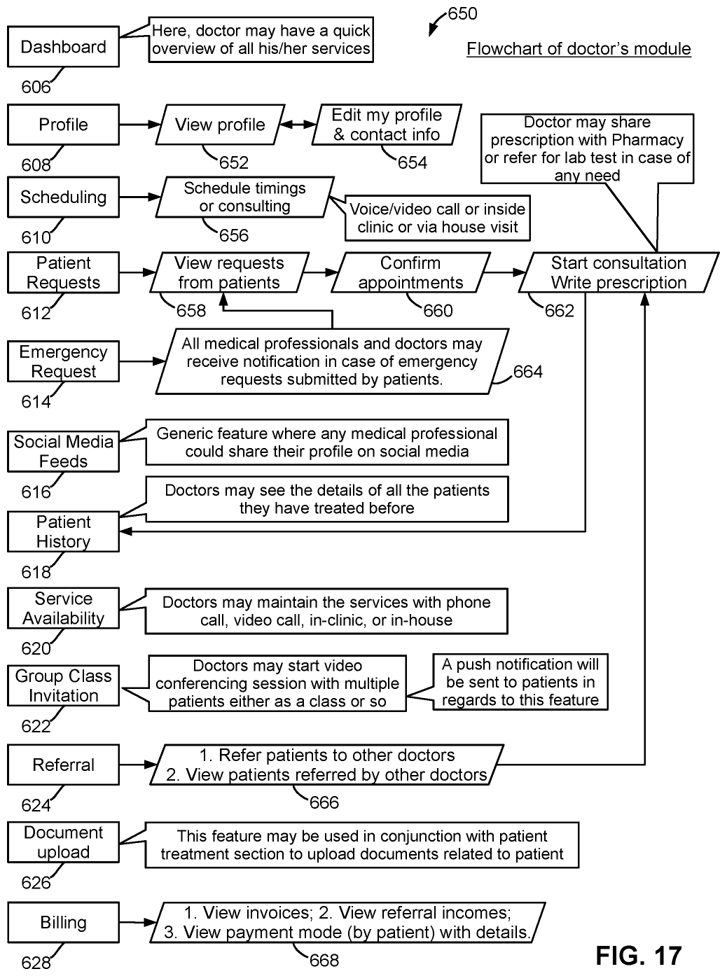

FIGS. 16A and 16B show a list of functions that a health-professional module 600 of the personalized health management system (i.e., a combination of the doctor's app 208 and corresponding program modules on the server 102) may provide to the doctor in some embodiments. FIG. 17 shows a flowchart 650 of the health-professional module 600.

As shown, the health-professional module 600 provides a registration function 602 for collecting various information of the health professional such as name, office phone number, office address, email address, office fax number, address, clinic name, designation and/or qualifications, profession (e.g., family, physio, cardio, and the like), license number, province, active license in the province, Brief description of the health professional, consent note, availability, fee schedule, clinic availability, waiting time in clinic, schedule services provided by the health professional, social media feeds, photo of the health professional, number of staff, signature (which is required as doctor signing on prescription is mandatory), and/or the like.

After registration 602, the doctor is then allowed to log-in (604) to the system 100. A dashboard 606 is then displayed via the doctor's app 208 which comprises various icons for the services provided by the system 100.

The doctor may use the dashboard 606 to conduct various tasks. For example, the doctor may view his/her profile 608 (e.g., photo, contact information, profession, and/or the like) stored in the system 100 (step 652 of FIG. 17) and edit the profile (step 654). For ease of use, the doctor's app 208 may comprise a “Profile” tab or menu item on the UI displayed thereon.

The doctor may use the dashboard 606 to schedule his/her time 610 for the services he/she provides (such as video consultancy, in-clinic consultancy, on phone or in-house visit, and/or the like) (step 656). For ease of use, the doctor's app 208 may comprise a “Schedule” tab or menu item on the UI displayed thereon.

The doctor may use the dashboard 606 to process patient's request 612 for consultancy. For example, the doctor may view requests received from patients (step 658), confirm appointments with patients (step 660), and start consultation and write prescriptions (step 662). For ease of use, the doctor's app 208 may comprise a “Patient Requests” tab or menu item on the UI displayed thereon.

The doctor may use the dashboard 606 to receive and process patient's emergency requests 614. For example, if the patient 202 needs consultancy immediately, the patient's app 204 may send notification to available health professional, and the health professional may respond using the dashboard 606. In these embodiments, all medical professionals and doctors may receive a notification when an emergency request is submitted by the patient 202 (step 664). For ease of use, the doctor's app 208 may comprise an “Emergency Request” tab or menu item on the UI displayed thereon.

The doctor may use the dashboard 606 to share social media feeds 616 such as the doctor's profile or a portion thereof. For ease of use, the doctor's app 208 may comprise a “Social Media” tab or menu item on the UI displayed thereon.

The doctor may use the dashboard 606 to review the medical history 618 of the patient 202 (after the patient 202 gives the doctor a permission). The doctor may be allowed to review the details of all the patients they have previously treated. For ease of use, the doctor's app 208 may comprise a “Medical History” tab or menu item on the UI displayed thereon.

The doctor may use the dashboard 606 to decide the services 620 he/she would like to provide, e.g., in-clinic, in-house, phone call, or video-conference. For ease of use, the doctor's app 208 may comprise a “Service Availability” tab or menu item on the UI displayed thereon.

The doctor may use the dashboard 606 to send invitation 622 to a group of patients and video-conference with the group of patients. In particular the doctor may start video conferencing session with a plurality of patients. A push notification regarding the group class invitation 622 may be sent to patients. For ease of use, the doctor's app 208 may comprise a “Group Class Invitation” tab or menu item on the UI displayed thereon.

The doctor may use the dashboard 606 to refer the patient 202 to another health professional 624 (step 666) and the two health professionals may interact with each other via the live-chat module 246 to discuss the patient's health conditions. The doctor may also review patients referred by other doctors. The doctor may later edit or change the referred doctor. For ease of use, the doctor's app 208 may comprise a “Referral” tab or menu item on the UI displayed thereon. In some embodiments, the doctor may send referral to lab test.

The doctor may use the dashboard 606 to upload the referral documents/patient charts/patient report and/or other documents 626 to the system 100 such as to the health database 232 thereof. For ease of use, the doctor's app 208 may comprise an “Upload Files” tab or menu item on the UI displayed thereon. In some embodiments, this function may be used in conjunction with patient treatment section to upload documents relevant to the patient.

The doctor may use the dashboard 606 to bill the patient 202 to the patient's financial account 628 such as the health account, insurance account, credit card, or other suitable account. In particular, the doctor may review invoices, review referral incomes, and view payment mode (by patient) with details (step 668). For ease of use, the doctor's app 208 may comprise a “Billing” tab or menu item on the UI displayed thereon.

In some embodiments, the doctor may use the dashboard 606 to share prescriptions with other health professionals such as pharmacists as needed. For ease of use, the doctor's app 208 may comprise a “Share” tab or menu item on the UI displayed thereon.

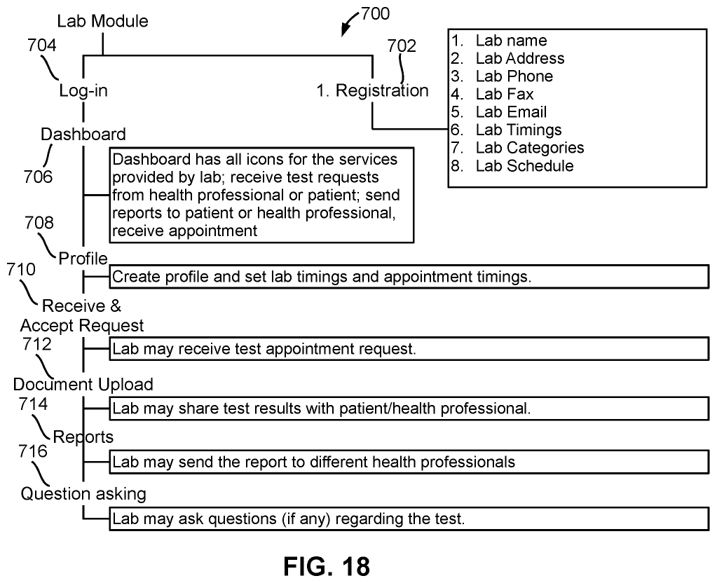

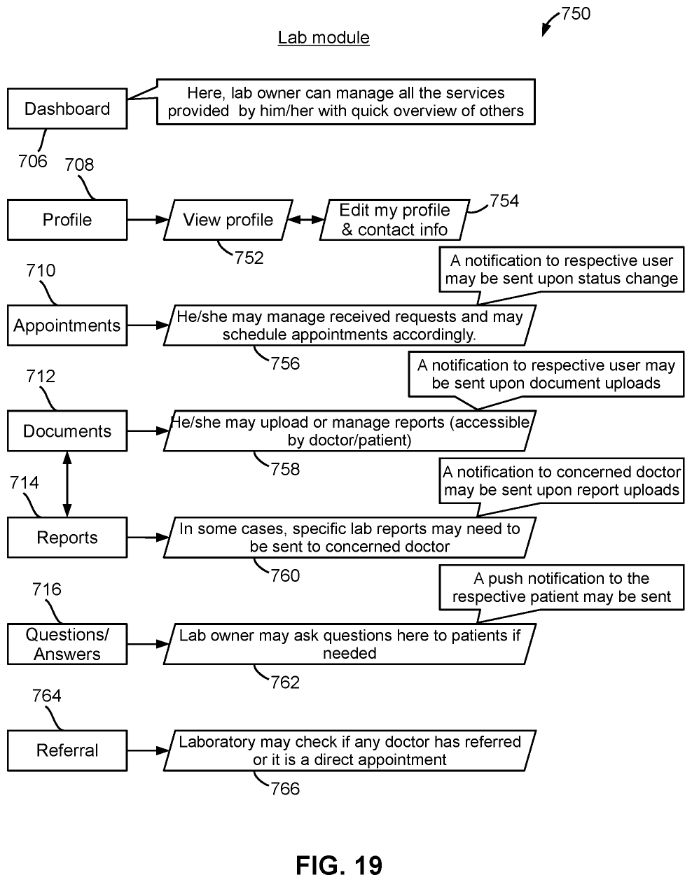

In some embodiments, the system 100 may also comprise a lab module for facilitating lab tests. FIG. 18 shows a list of functions that the lab module 700 may provide to a lab staff in these embodiments. FIG. 19 shows a flowchart 750 of the lab module 700.

As shown, the lab module 700 provides a registration function 702 for collecting various information of the lab staff such as lab name, lab address, lab phone number, lab fax number, lab email address, lab hours, lab categories, lab schedule, and/or the like.

After registration 702, the lab staff is then allowed to log-in (704) to the system 100. A dashboard 706 is then displayed via a lab-staff app (not shown) which comprises various icons for the services provided by the system 100.

The lab staff may use the dashboard 706 to conduct various tasks. For example, the lab staff may view his/her profile 708 (e.g., photo, contact information, profession, and/or the like) stored in the system 100 (step 752 of FIG. 19) and edit the profile (step 754). For ease of use, the app used by a lab staff (denoted as “lab app” hereinafter) may comprise a “Profile” tab or menu item on the UI displayed thereon.

The lab staff may use the dashboard 706 to receive test appointment requests 710 and arrange appointments accordingly (step 756). A notification to respective user may be sent upon appointment status changes. For ease of use, the lab app may comprise an “Appointment” tab or menu item on the UI displayed thereon.

The lab staff may use the dashboard 706 to upload the referral test results and/or other documents 712 to the system 100 such as to the health database 232 thereof (step 758) and share test results with the patient 202 and the health professional. A notification to respective user may be sent upon document uploads. For ease of use, the lab app may comprise an “Upload Files” tab or menu item on the UI displayed thereon.

The lab staff may use the dashboard 706 to send reports 714 to different health professionals. The reports may be files uploaded at step 758. In some cases, specific lab reports may need to be sent to concerned doctor (step 760). A notification to concerned doctor may be sent upon report uploads. For ease of use, the lab app may comprise a “Reports” tab or menu item on the UI displayed thereon.

The lab staff may use the dashboard 706 to answer questions 716 (step 762). A push notification to the respective patient may be sent. For ease of use, the lab app may comprise an “Answer Questions” tab or menu item on the UI displayed thereon.

The lab staff may use the dashboard 706 to process referrals 764. The lab staff may check if any doctor has referred or it is a direct appointment. For ease of use, the lab app may comprise a “Referral” tab or menu item on the UI displayed thereon.

In some embodiments, the lab staff may use the dashboard 706 to set lab hours, and/or other tasks.

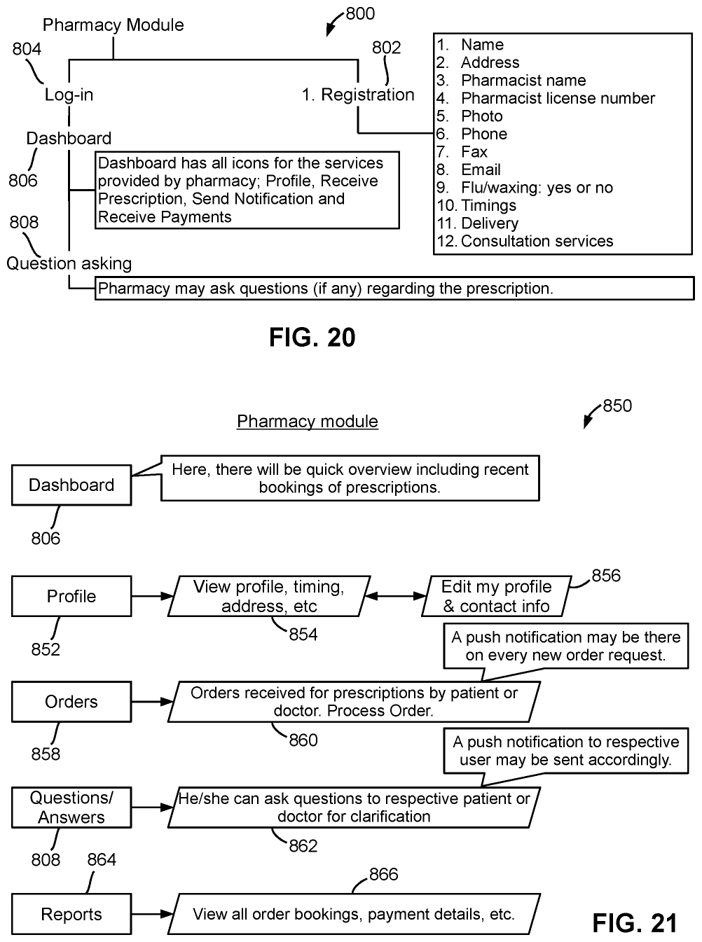

In some embodiments, the system 100 may further comprise a pharmacy module. FIG. 20 shows a list of functions that the pharmacy module 800 may provide to a pharmacy staff (e.g., a pharmacist or an employee of the pharmacy) in these embodiments. FIG. 21 shows a flowchart 850 of the pharmacy module 800.

As shown, the pharmacy module 800 provides a registration function 802 for collecting various information of the pharmacy staff such as name, address, pharmacist's name, pharmacist's license number, photo, phone number, fax number, email address, flu/waxing (yes or no), work hours, delivery information, consultation services, and/or the like.

After registration 802, the pharmacy staff is then allowed to log-in (804) to the system 100. A dashboard 806 is then displayed via a pharmacy-staff app (not shown) which comprises various icons for the services provided by the system 100.

The pharmacy staff may use the dashboard 806 to conduct various tasks. For example, the pharmacy staff may view his/her profile 852 (e.g., photo, contact information, timing, address, and/or the like) stored in the system 100 (step 854 of FIG. 21) and edit the profile (step 856). For ease of use, the pharmacy-staff app may comprise a “Profile” tab or menu item on the UI displayed thereon.

In these embodiments, orders 858 may be received for prescriptions by patient or doctor. The pharmacy staff may use the dashboard 806 to process the orders 858 (step 860). A push notification may be sent to the pharmacy staff on every new order request. For ease of use, the pharmacy-staff app may comprise an “Order” tab or menu item on the UI displayed thereon.

If needed, the pharmacy staff may use the dashboard 806 to ask the patient 202 or the doctor questions 808 regarding the prescription (step 862). For ease of use, the pharmacy-staff app may comprise a “Questions” tab or menu item on the UI displayed thereon. The pharmacy staff may use the dashboard 806 to prepare reports 864 such as viewing all order bookings, viewing payment details, and/or the like (step 866). For ease of use, the pharmacy-staff app may comprise a “Report” tab or menu item on the UI displayed thereon.

In some embodiments, the pharmacy staff may use the dashboard 806 to receive payments.

In some embodiments, the AI platform 206 may be functionally coupled to one or more geo-location sensors for estimating user location as needed to provide geo-coordinates to doctors and/or healthcare providers.

In case of emergency, the reader sends geospatial information to the nearest emergency services.

Although embodiments have been described above with reference to the accompanying drawings, those of skill in the art will appreciate that variations and modifications may be made without departing from the scope thereof as defined by the appended claims.

1. A network system for monitoring health conditions of one or more patients, the system comprising:
one or more health-monitoring data sources for acquiring health-related data of the one or more patients, said one or more data sources comprising at least one personalized health-monitoring device;
one or more first client-computing devices associated with and used by the one or more patients;
one or more second client-computing devices associated with and used by one or more health professionals;
one or more server computers;
a secured database; and
a network functionally coupled to the one or more health-monitoring data sources, the one or more first client-computing devices, the one or more second client-computing devices, the one or more server computers, and the secured database;
wherein the one or more first client-computing devices, the one or more second client-computing devices, and the one or more server computers are configured for:
collecting health-related data acquired by the one or more health-monitoring data sources;
storing the collected health-related data in the secured database as electronic medical records (EMRs);
automatically analyzing the collected health-related data and assessing the health conditions of the one or more patients using an artificial intelligence (AI) platform for obtaining health-analysis results and health and medical advices;
based on the obtained health-analysis results and assessed health conditions, directing each of the one or more patients for one of managing their health conditions at home, visiting a clinic for health management, and visiting a hospital or emergency room for health management; and
updating the EMRs.

2. The system of claim 1, wherein the one or more first client-computing devices, the one or more second client-computing devices, and the one or more server computers are further configured for:
receiving input from the one or more second client-computing devices; and
revising the obtained health-analysis results and health and medical advices based the received input.

3. The system of claim 1, wherein the one or more first client-computing devices, the one or more second client-computing devices, and the one or more server computers are further configured for:
storing a portion of the EMRs into a personal-data database of each of the one or more first client-computing devices, the portion of the EMRs being related to the patient associated with said first client-computing device.

4. The system of claim 1, wherein each of the one or more first client-computing devices comprises a first live-chat module, and each the one or more second client-computing devices comprising a second live-chat module; and wherein the first and second live-chat modules are communicative with each other via the network.

5. The system of claim 1, wherein the AI platform is configured for using a neural network to process and analyze the collected health-related data.

6. The system of claim 5, wherein the collected health-related data comprises a personalized health questionnaire.

7. The system of claim 1, wherein the AI platform is configured for monitoring physical activity and physiological health indicators of at least one of a heart rate, a heart rhythm, a blood pressure, a breathing pattern, a blood glucose level, and a blood oxygen saturation.

8. The system of claim 1, wherein the AI platform is configured for one or more of evaluating the medical conditions of the one or more patients, providing medical diagnoses thereto, providing health management thereto, providing medical advices thereto, and communicating the health or medical information thereof to one or more health professionals and/or emergency-response professionals, based on the analysis of the collected health-related data and assessment of the health conditions of the one or more patients.

9. The system of claim 1, wherein the at least one personalized health-monitoring device each comprises a portable reader and a cartridge for receiving and analyzing samples of bodily fluids and/or tissue if a respective one of the one or more patients.

10. The system of claim 9, wherein the cartridge comprises a nanostructured sensing surface for collecting and amplifying a biomolecular binding-signal of said samples of bodily fluids and/or tissue.

11. A method for monitoring health conditions of one or more patients, the method comprising:
collecting health-related data of the one or more patients from one or more health-monitoring data sources, said one or more data sources comprising at least one personalized health-monitoring device;
storing the collected health-related data in a secured database as electronic medical records (EMRs);
automatically analyzing the collected health-related data and assessing the health conditions of the one or more patients using an artificial intelligence (AI) platform for obtaining health-analysis results and health and medical advices;
based on the obtained health-analysis results and assessed health conditions, directing each of the one or more patients for one of managing their health conditions at home, visiting a clinic for health management, and visiting a hospital or emergency room for health management; and
updating the EMRs.

12. The method of claim 11 further comprising:
receiving input from the one or more second client-computing devices; and
revising the obtained health-analysis results and health and medical advices based the received input.

13. The method of claim 11 further comprising:
storing a portion of the EMRs into a personal-data database of each of the one or more first client-computing devices, the portion of the EMRs being related to the patient associated with said first client-computing device.

14. The method of claim 11 further comprising:
providing a first live-chat module to each of the one or more patients;
providing a second live-chat module to each of one or more health professionals; and
establishing communication between at least one of the first live-chat modules and at least one of the second live-chat modules via a network.

15. The method of claim 11 further comprising:
using a neural network in the AI platform to process and analyze the collected health-related data.

16. The method of claim 15, wherein the collected health-related data comprises a personalized health questionnaire.

17. The method of claim 11 H further comprising:
using the AI platform for monitoring physical activity and physiological health indicators of at least one of a heart rate, a heart rhythm, a blood pressure, a breathing pattern, a blood glucose level, and a blood oxygen saturation.

18. The method of claim 11, wherein the AI platform is configured for one or more of evaluating the medical conditions of the one or more patients, providing medical diagnoses thereto, providing health management thereto, providing medical advices thereto, and communicating the health or medical information thereof to one or more health professionals and/or emergency-response professionals, based on the analysis of the collected health-related data and assessment of the health conditions of the one or more patients.

19. The method of claim 11, wherein the at least one personalized health-monitoring device each comprises a portable reader and a cartridge for receiving and analyzing samples of bodily fluids and/or tissue if a respective one of the one or more patients; and
wherein said collecting the health-related data comprises:
receiving the samples of bodily fluids and/or tissue using the cartridge;
analyzing the received samples of bodily fluids and/or tissue using the portable reader; and
collecting the health-related data from the portable reader.

20. The method of claim 19, wherein the cartridge comprises a nanostructured sensing surface for collecting and amplifying a biomolecular binding-signal of said samples of bodily fluids and/or tissue.
